(19) United States
(12) Patent Application Publication
(10) Pub. No.: US 2024/0386999 A1
(43) Pub. Date: Nov. 21, 2024

(54) DENOISING AND GENE FILTERING FOR SINGLE CELL SEQUENCING DATA USING GRAPH MINING

(71) Applicant: Accenture Global Solutions Limited, Dublin (IE)

(72) Inventors: Maziyar Baran Pouyan, Petaluma, CA (US); Aldan Creo Marino, Dublin (IE)

(21) Appl. No.: 18/658,251

(22) Filed: May 8, 2024

Related U.S. Application Data
(60) Provisional application No. 63/502,172, filed on May 15, 2023.

(51) Int. Cl. G16B 30/00; G06F 30/20; G16B 40/00; G16B 45/00

(52) U.S. Cl. CPC G16B 30/00; G06F 30/20; G16B 40/00; G16B 45/00

(57) ABSTRACT

Improved denoising and gene filtering systems and methods for single cell sequencing datasets using graph mining. The system encodes the dataset into a graph data structure including cell nodes, gene nodes, and edges representing the gene expression levels measured for each cell node. The graph data structure is then processed using a node embedding algorithm and a link prediction model to identify edges that should have been captured in the dataset. In addition, the graph data structure can be processed using a community detection algorithm to identify nodes of highly associated communities. Specificity gene scores can then be computed based on the communities to filter genes with greater accuracy.

### CROSS-REFERENCE TO RELATED APPLICATION

This application claims the benefit of U.S. Provisional Patent Application Ser. No. 63/502,172 filed on May 15, 2023 and titled “Identifying and Quantifying Relationships Amongst Cells and Genes”, the disclosure of which is incorporated by reference herein in its entirety.

### TECHNICAL FIELD

The present disclosure generally relates to improving accuracy of denoising and gene filtering techniques for single cell sequencing datasets using graph mining.

### BACKGROUND

Traditionally, ribonucleic acid (RNA) has been analyzed by bulk sequencing, which involves analyzing groups of cells rather than individual cells. The advancement of single cell sequencing improves the ability to identify more granular properties of individual cells. Single cell sequencing can currently be used to measure the genome (scDNA-seq), the DNA-methylome or the transcriptome (scRNA-seq), proteomic, and ATAC-seq of each cell of a population.

However, single cell denoising remains an open problem in this area. The accuracy of the data representing each gene's actual level of expression in each cell is inconsistent. For example, noise due to amplification and dropout may obstruct analyses, so scalable denoising methods for increasingly large but sparse scRNA-seq data are needed. Approaches have been proposed to increase accuracy, yet they carry the drawback of causing extra and non-biological artifacts into the data. In addition, current gene filtering techniques for scRNA-seq data are unable to ensure that low expression genes that may nevertheless be biologically meaningful are retained.

There is a need in the art for a system and method that addresses the shortcomings discussed above.

### SUMMARY

The proposed systems and methods describe improved denoising and data filtering techniques for use with single cell sequencing datasets. During measurement of the transcriptome across thousands of cells, there is frequently a great deal of technical noise connected with the measurements (e.g., Unique Molecular Identifier (UMI) counts). More specifically, the low RNA capture rate during single cell sequencing leads to a failure of detection of an expressed gene resulting in a “false” zero count observation. These non-biological zeros reflect the loss of information about truly expressed genes due to the inefficiencies of the technologies employed from sample collection to sequencing. Because true zeros (or biologically correct zeros) exist, it can be difficult to determine whether a measurement of zero is true or if the measurement of zero is an error made by equipment while attempting to capture gene expression. The disclosed systems address this deficiency by incorporating the features of graph mining to identify relationships (expression levels) between genes and cells where an incorrect zero has been measured. The systems encode the original dataset into a graph data structure that can then be passed to a node embedding algorithm. The vector representations generated are then processed by a link prediction model to determine which gene expression values that had been logged as zeros in the dataset should be non-zero.

Furthermore, the proposed embodiments are effective in reducing over-filtering during analyses of the single cell sequencing datasets by using the graph mining techniques described herein. For example, the graph data structure can be passed to a community detection algorithm to identify highly related nodes. This information can then be used to compute specificity gene scores which enable a ranking of the genes to serve as an alternative reference for filtering or excluding some of the less relevant or meaningful data.

In one aspect, a method of denoising a single cell sequencing dataset is disclosed. The method includes a first step of receiving a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell. A second step includes encoding the gene expression matrix in a first graph data structure that includes a group of nodes including: (a) a set of cell nodes corresponding to the plurality of cells, (b) a set of gene nodes corresponding to the plurality of genes, and (c) edges connecting each cell node with any gene node where the gene associated with that gene node has a non-zero expression level in the cell associated with that cell node. In addition, a third step includes passing the first graph data structure through a node embedding algorithm to compute an output including a vector representation of each node and each edge, and a fourth step includes passing the output through a link prediction model to predict the existence of non-zero gene expression values where a zero gene expression value had originally been incorrectly identified in the single cell sequencing dataset.

In another aspect, a method of improving accuracy of gene filtering and reducing technical noise in single cell sequencing datasets is disclosed. The method includes a first step of receiving a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell. A second step includes encoding the gene expression matrix in a first graph data structure that includes a group of nodes including: (a) a set of cell nodes corresponding to the plurality of cells, (b) a set of gene nodes corresponding to the plurality of genes, and (c) edges connecting each cell node with any gene node where the gene associated with that gene node has a non-zero expression level in the cell associated with that cell node. A third step includes passing the first graph data structure through a community detection algorithm to identify a group of communities of more densely interconnected nodes. In addition, a fourth step includes calculating a specificity gene score for each gene node to generate a set of specificity gene scores, and a fifth step includes excluding those gene nodes with a specificity gene score less than at least 75% of the set of specificity gene scores.

In another aspect, a system for denoising a single cell sequencing dataset is disclosed. The system includes one or more computers and one or more storage devices that are operable, when executed by the one or more computers to: (1) receive a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell; (2) encode the gene expression matrix in a first graph data structure including a group of nodes including a set of cell nodes corresponding to the plurality of cells and a set of gene nodes corresponding to the plurality of genes, and edges connecting each cell node with any gene node where the gene associated with that gene node has a non-zero expression level in the cell associated with that cell node; (3) pass the first graph data structure through a node embedding algorithm to compute an output including a vector representation of each node and each edge; and (4) pass the output through a link prediction model to predict the existence of non-zero gene expression values where a zero gene expression value had originally been incorrectly identified in the single cell sequencing dataset.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1A is a sample gene expression matrix is shown to introduce a technical problem associated with single cell sequencing datasets, according to an embodiment;

FIG. 1B is a schematic diagram of an overview of methods of extracting gene data, according to an embodiment;

FIG. 1C is a schematic diagram of an overview of a single cell multi-omics and further downstream processes, according to an embodiment;

FIG. 2 depicts another gene expression matrix, with data entries representing a single cell sequencing dataset and a graph encoding the data entries, according to an embodiment;

FIG. 3 is a schematic diagram showing the graph of FIG. 2 being passed through a node embedding algorithm, according to an embodiment;

FIG. 4 is a schematic diagram depicting the output of the node embedding algorithm being passed through a link prediction model for purposes of identifying inappropriate zero values in the gene expression matrix, according to an embodiment;

FIG. 5 is a schematic diagram showing the data matrix being encoded as a graph for purposes of improving gene filtering, according to an embodiment;

FIG. 6 is a schematic diagram of the graph being passed through a community detection algorithm for computing weighted centrality measurements of the gene nodes, according to an embodiment;

FIG. 7 is a schematic diagram depicting example environments and components by which systems and/or methods, described herein, may be implemented, according to an embodiment; and

FIG. 8 is a flow chart presenting a method of denoising a single cell sequencing dataset, according to an embodiment.

### DESCRIPTION OF EMBODIMENTS

Single-cell RNA-Sequencing (scRNA-Seq) enables the simultaneous measurement of the transcriptome across thousands of cells from complex tissues and entire organs. Such single-cell analyses can allow researchers to uncover new and potentially unexpected biological discoveries relative to traditional profiling methods that assess bulk populations. Single-cell RNA sequencing, for example, can reveal complex and rare cell populations, uncover regulatory relationships between genes, and track the trajectories of distinct cell lineages in development.

However, single-cell RNA-Seq measurements are commonly affected by high levels of technical noise, posing challenges for data analysis and visualization. Despite improvements in measuring technologies, various technical factors, including amplification bias, cell cycle effects, library size differences, and especially low RNA capture rate can lead to substantial noise in scRNA-seq experiments. For example, droplet-based scRNA-seq technologies can profile up to millions of cells in a single experiment. Unfortunately, these technologies are particularly sparse due to relatively shallow sequencing. Overall, these types of technical factors introduce substantial noise, which may corrupt the underlying biological signal and obstruct analysis.

Furthermore, analyses of single-cell RNA sequencing data typically involve gene filtering as an initial step. Because gene filtering is performed early on, this step can drastically change the performance of downstream analyses. Yet several studies have indicated that some cell types are under- or over-represented in the final datasets due to artificial filtering. For example, traditional gene filtering techniques for scRNA-seq data do not reliably identify rare cell populations. Conventional approaches seeking to resolve this issue tend to remove low expressed genes, even though these low expressed genes can be critical for researchers in discovering rare cell types in various diseases such as tumors.

The proposed systems and methods offer a denoising strategy for Single Cell Gene Expression Retrieval based on graph mining (e.g., Cell-Gene Relation Graphs (CGRG). As a general matter, denoising refers to the process of determining each gene's actual level of expression in each cell. As will be described in greater detail below, the proposed graph-based single cell denoising techniques can accurately correct the data produced by scRNA-seq experiments, particularly with respect to maintaining true biological zeros at zero while correctly predicting the technical ones, without adding extra information or non-biological artifacts into the data. In contrast to conventional approaches, the proposed systems employ graph mining techniques to substantially improve the quality and consistency of the zero expression measurements in the data.

Furthermore, as will be described below, graph mining can also be used to avoid the undesirable removal of low expressed genes. For example, in different embodiments, a gene filtering technique that uses Cell-Gene Relation Graphs (CGRG) can retain low expression but informative genes for scRNA-Seq data to enable identification of biologically meaningful rare cell populations.

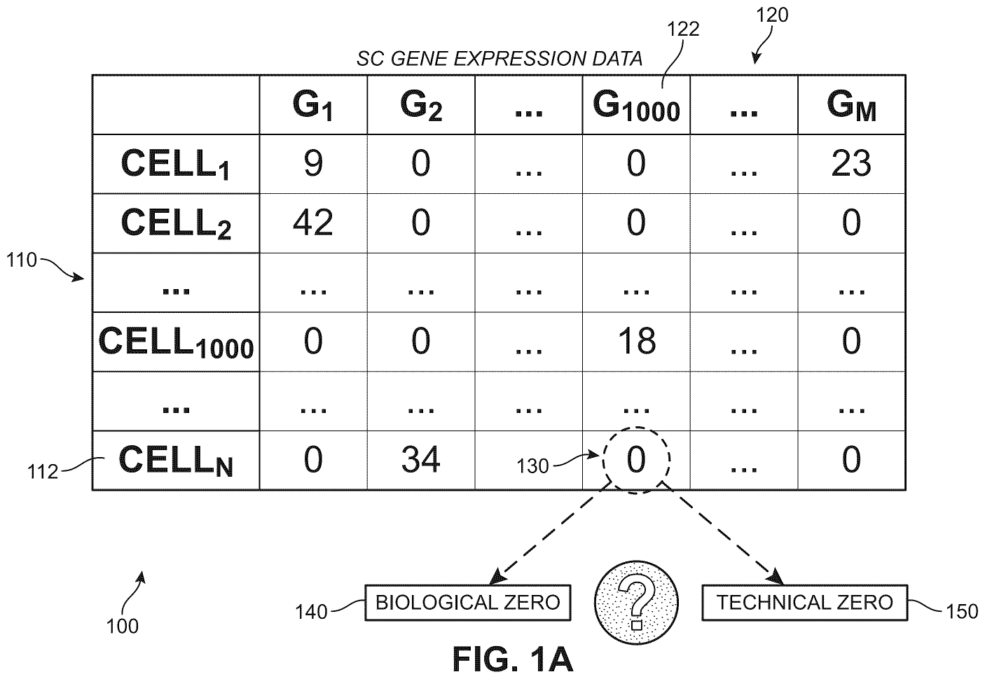

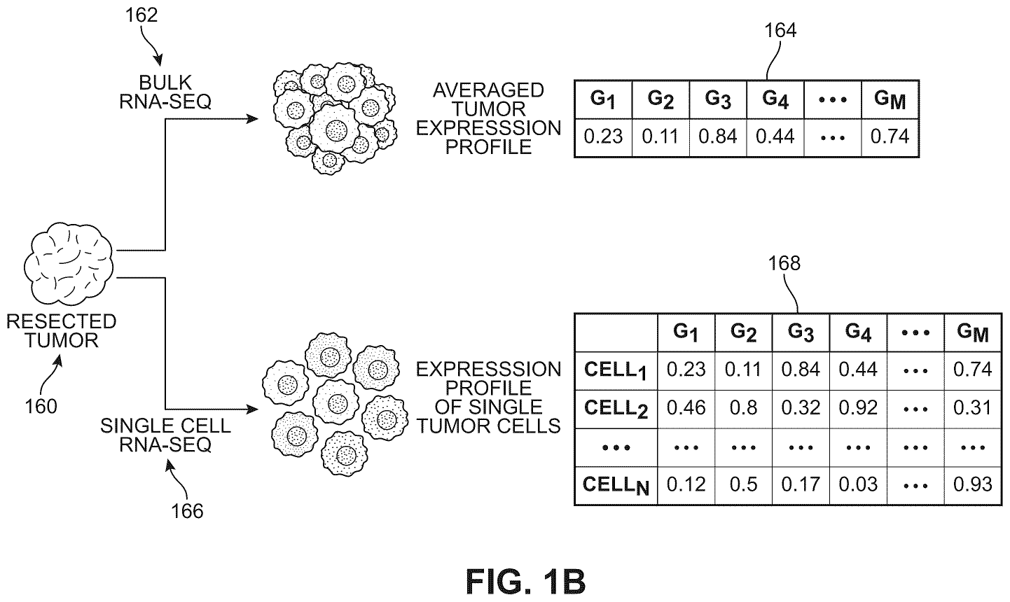

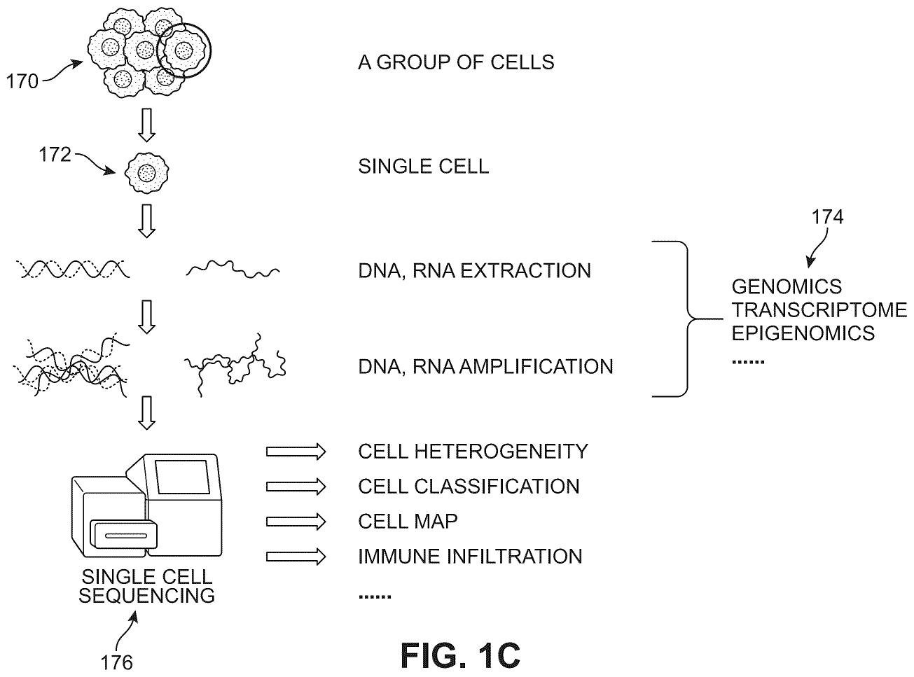

Referring now to FIG. 1A, for purposes of introduction to a critical technical problem associated with scRNA-seq data analyses, a sample single cell gene expression matrix (“sample matrix”) 100 is provided. In general, only a small portion of the mRNA molecules present in each cell are detected by single-cell RNA-Seq techniques. As a result, there is frequently a great deal of technical noise connected with the measurements (e.g., Unique Molecular Identifier (UMI) counts) that are seen for each gene and each cell. UMI counts represent the absolute number of observed transcripts (per gene, cell or sample). The output of a Single-cell RNA sequencing (scRNA-seq) can be represented as presented in sample matrix 100, where rows 110 refer to specific cells, and columns 120 refer to the specific expression for that gene in that cell.

It can be observed that-in large part due to sparsity—most of the entries are reflected by zero values. More specifically, the low RNA capture rate during single cell sequencing leads to a failure of detection of an expressed gene resulting in a “false” zero count observation, also defined as dropout event. It is at this juncture that it becomes imperative to recognize there is an important distinction between “false” and “true” zero counts. True zero counts represent the lack of expression of a gene in a specific cell-type: a true cell-type-specific expression. Therefore, not all zeros in scRNA-seq data can be considered missing values. A universal analytical challenge for scRNA-seq data generated by any protocol is the vastly high proportion of genes with zero expression measurements in each cell. Excess zeros can bias the estimation of gene expression correlations and hinder the capture of gene expression dynamics from scRNA-seq data.

Zero measurements in scRNA-seq data have two sources: (a) biological and (b) non-biological. While biological zeros carry meaningful information about cell states, non-biological zeros represent missing values artificially introduced during the generation of scRNA-seq data. Non-biological zeros include technical zeros, which occur during the preparation of biological samples for sequencing, and sampling zeros, which arise due to limited sequencing depths. Non-biological zeros have typically been viewed as impediments to the full and accurate interpretation of cell states and the differences between them. It is worth noting that biological and non-biological zeros are hardly distinguishable in scRNA-seq data without biological knowledge or spike-in control.

Thus, a biological zero is significant: it is defined as the true absence of a gene's transcripts or messenger RNAs (mRNAs) in a cell. Biological zeros occur for two reasons: (1) many genes are unexpressed in a cell and cells of distinct types have different genes expressed-a fact that results in the diversity of cell types; and (2) many genes undergo a bursty process of transcription (i.e., mRNA synthesis); that is, these genes are not transcribed constantly but intermittently, a well-known phenomenon in gene regulation. Due to the stochasticity of specific transcription factors (TFs) binding, a gene switches between active and inactive states, and its transcription only occurs during the active state. Depending on the gene's switching rates between the active and inactive states, transcription rate, and degradation rate, the resulting distribution may exhibit a mode near zero, which makes it appear that the gene expresses no mRNA at a particular time, in a large number of cells.

On the other hand, non-biological zeros reflect the loss of information about truly expressed genes due to the inefficiencies of the technologies employed from sample collection to sequencing. Unlike biological zeros, non-biological zeros refer to the zero expression measurements of genes with transcripts in a cell. As noted above, there are two types of non-biological zeros: (a) technical zeros, which arise from library-preparation steps before sequencing, and (b) sampling zeros, which result from a limited sequencing depth. For example, if a gene's mRNA transcripts in a cell are not converted into cDNA molecules (cDNAs), the gene would falsely appear as non-expressed in that cell in the sequencing library, resulting in a technical zero in scRNA-seq data. Sampling zeros typically occur due to a constraint on the total number of reads sequenced, i.e., the sequencing depth, which is determined by the experimental budget and sequencing machine. During sequencing, cDNAs are randomly captured (“sampled”) and sequenced into reads. Hence, a gene with fewer cDNAs is more likely to be undetected due to this random sampling. If undetected, the gene's resulting zero read count is a “sampling zero.”

As a general matter, in statistics, missing data values are typically imputed. In this process, missing values are substituted for values either randomly or by adapting to the data structure, to improve statistical inference or modeling. Due to the non-trivial distinction between true and false zero counts, classical imputation methods with defined missing values are not suitable for scRNA-seq data and should not be used. Instead, denoising strategies can be used to delineate signal from noise in imaging. Due to a large number of technical zeros that may be generated during single cell sequencing protocols, directly processing the raw data may be detrimental to downstream analysis, such as clustering and visualization. Denoising therefore improves the process of finding gene expression values by determining each gene's actual level of expression in each cell.

For example, with respect to a first zero value 130 logged for a “CellN” 112 and a “Gene G1000” listed in the sample matrix 100, it is uncertain whether the zero obtained is actually biological (true zero) 140, or non-biological (false zero) 150. The proposed techniques enable reconstructions of the correct value of zeros that are non-biological false zeros (technical noise), while maintaining those zero value data entries that corresponded to biological true zeros. In other words, the disclosed embodiments offer a single cell denoising method based on graph mining that accurately corrects the data so that true biological zeros are maintained at zero while non-biological false zeros are identified and updated to their actual biological value.

For purposes of context to the reader, FIG. 1B presents a schematic diagram of an overview of methods of extracting gene data to convey how single cell sequencing yields much more detailed information than bulk sequencing. In this example, a resected tumor sample 160 may undergo bulk RNA sequencing 162 to produce an averaged tumor expression profile. Table 164 shows an example of columns representing genes. The value for each column is an average gene expression value for all of the cells analyzed in resected tumor 160. Also in this example, the resected tumor sample may undergo single cell RNA sequencing 166 to produce an expression profile of single tumor cells. Table 168 shows an example of columns representing genes and rows representing individual cells from resected tumor sample 160. The value for each gene is specific to an individual cell. As discussed above, bulk sequencing produces an average genome, which is representative of broad strokes of a genome. Single cell sequencing produces genomes of individual cells that form a cell population. Table 164 next to table 168 demonstrates how much more information is extracted by single cell RNA sequencing than by bulk RNA sequencing. The advancement of single cell sequencing improves the ability to identify more granular properties of individual cells and to measure the RNA expression of a considerable amount of single cells simultaneously, resulting in greatly increasing the knowledge of cellular structure.

Embodiments may include single cell multi-omics and further downstream processes. FIG. 1C is a schematic diagram of an overview of a single cell multi-omics and further downstream processes, according to an embodiment. This example demonstrates how applying hyperdimensional computing and dimension reduction to extract properties from a sparse data set can be used in single cell multi-omics and further downstream processes. Single cell multi-omics can begin with single cell RNA sequencing, which can include collecting a group of cells 170, e.g., by resection. In some embodiments, the method may include collecting a human tissue sample. For example, a group of cells may be obtained from a human tissue sample. In some embodiments, the human tissue sample may be collected by resecting directly. In other embodiments, the human tissue sample may be collected by receiving an already-resected human tissue sample. Single cell RNA sequencing can further include isolating a single cell 172 from a cell population. The method may include isolating a single cell from the human tissue sample. In some embodiments, single cell RNA sequencing can include extracting, processing, and amplifying Deoxyribonucleic Acid (DNA) and RNA of each isolated cell to perform multi-omics 304, such as genomics, transcriptome, and epigenomics. The method may include performing single cell sequencing 176 to generate data that can be used to perform downstream processes, such as determining cell heterogeneity, cell classification, generating a cell map, and identifying immune infiltration. The information generated by single cell sequencing 176 can include zero gene expression values. As explained above, these zero values can be either a biological zero that is a true absence of a gene's expression in a cell or a non-biological zero artificially introduced during generation of the single cell sequencing dataset. The disclosed methods of denoising can determine biological zeros and values erroneously measured as zero during single cell sequencing. The disclosed graph mining techniques can reduce over-filtering during analyses of the single cell sequencing datasets.

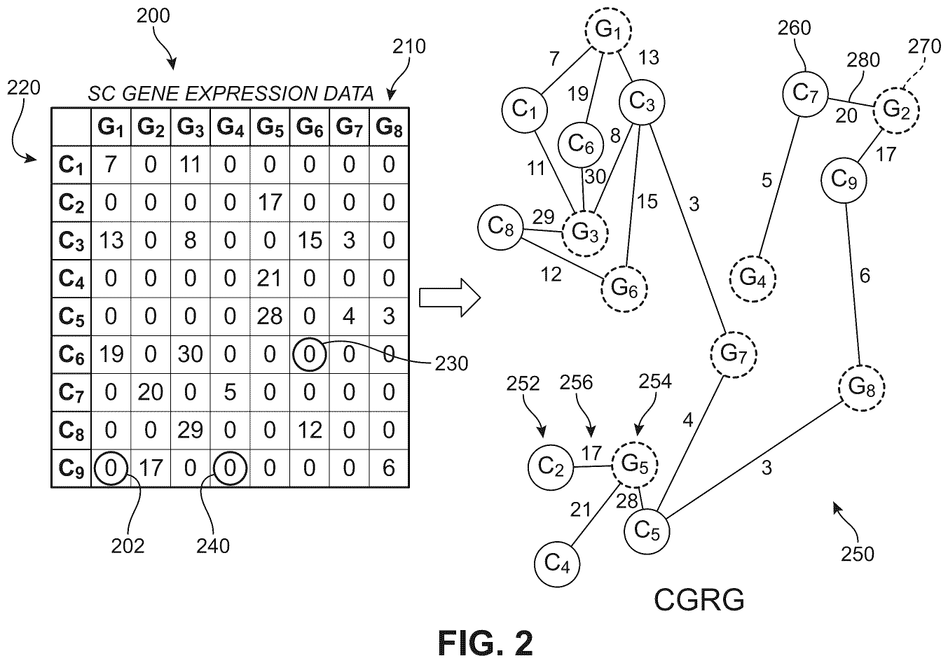

Moving now to FIGS. 2, 3, and 4, an embodiment of a denoising process based on graph mining is provided that addresses these deficiencies. In FIG. 2, an example of a single cell gene expression data matrix 200 is presented. The matrix 200 includes a plurality of rows 220, one row for each cell (e.g., C1, C2, C3, . . . . Cg), and a plurality of columns 210, one column for each of a specific gene whose expression level is being measured (e.g., G1, G2, G3, . . . . G8). It can be observed that the majority of data entries are logged as zero values. For example, at (C9, G1) there is a first zero entry 202, at (C9, G4) there is a second zero entry 240, and at (C6, G6) there is a third zero entry 230. At this stage, it is uncertain whether these zeros are true zeros or false zeros.

In different embodiments, a first stage of the proposed denoising process transforms the data from matrix 200 to a graph. In this initial stage, the cells and genes are encoded into a single graph with nodes representing distinct entities (i.e., each of the genes and each of the cells) and edges denoting connections between entities. One example of this is shown in FIG. 2. For purposes of this disclosure, this graph can be referred to as a first “Cell-Gene Relation Graph” (CGRG) 250. Using this technique, an edge 256 is made that extends between the different genes 254 and cells 252 if the gene is expressed in that cell. In addition, the weight of this edge is determined by the gene expression level (count value), which is shown as a number associated with that edge. As a specific example, a first cell 260 is shown connected by a first edge 280 to a first gene 270, where the count value associated with the first edge 280 has a value of 20.

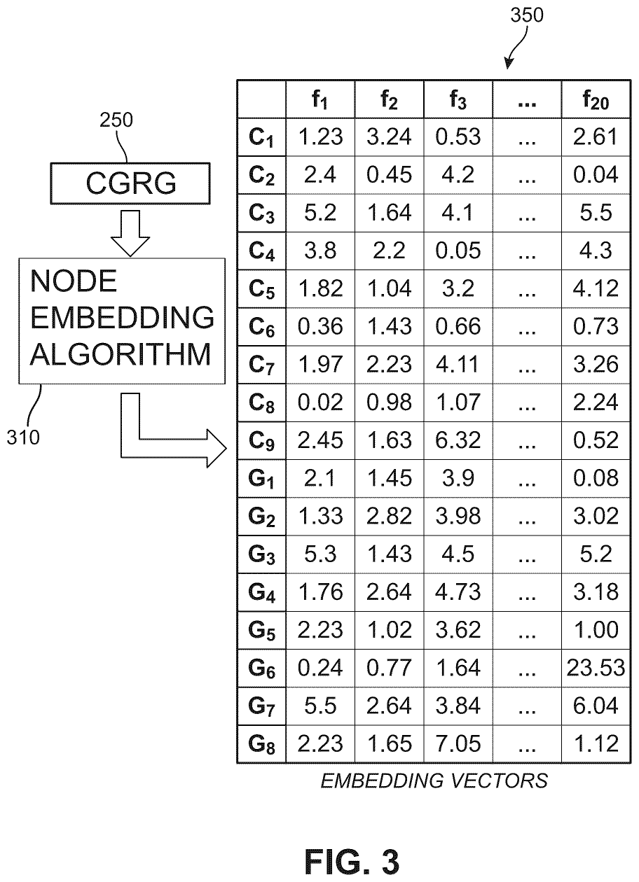

Referring next to FIG. 3, in a second stage, the first CGRG 250 can be passed through a node embedding algorithm 310 (e.g., Node2Vec, Fast Random Projection, NodePiece, etc.) 310. In general, node embedding algorithms compute a vector representation of each node and each vertex or edge based on random walks in the graph. In different embodiments, the algorithm 310 can extract an embedding vector for each cell and gene. An embedding vectors matrix (“embedding vectors”) 350 is presented in FIG. 3 that represents the vector representation output (f) of the node embedding algorithm on the first CGRG 250 for each cell (C) and gene (G).

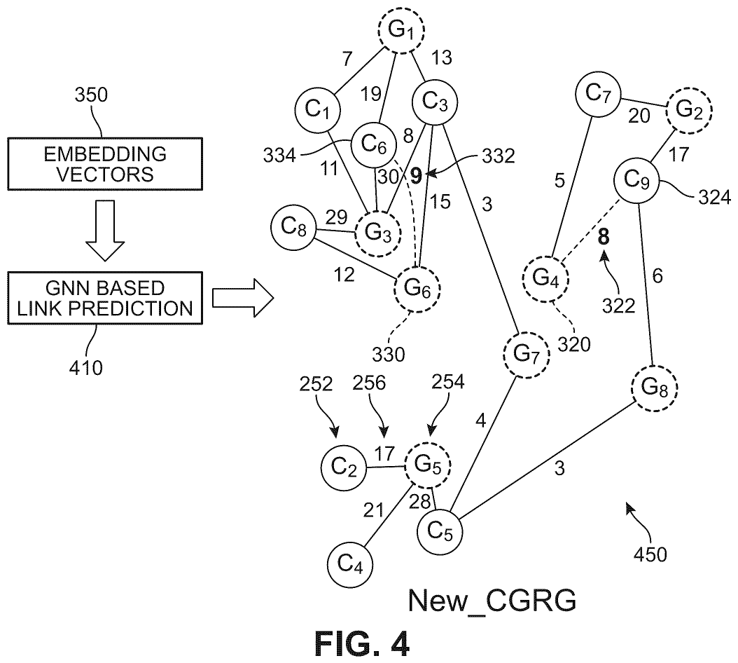

At a third stage, shown in FIG. 4, the data from the embedding vectors matrix 350 can then be passed through a Graph Neural Network (GNN)-based link prediction model 410 such as GraphSage. In different embodiments, the link prediction model 410 can include Poisson regression modeling techniques to identify potential links in the CGRC and predict gene expression values in the data. The link prediction model 410 can receive the input comprising data from the embedding vectors matrix 350 to predict the existence of an edge between two arbitrary nodes in a graph. In some embodiments, the GNN can be used to iteratively update node representations by aggregating the representations of node neighbors and their representation from the previous iteration.

The link prediction model 410 can output an updated first CGRG 450, an example of which is depicted in FIG. 4. It can be appreciated that there are now two new links that have been recovered (as compared to the first CRCG 250 of FIG. 2). These two new edges represent the recovery of two missing biological zeros. More specifically, there is now a second edge 322 extending between a second gene 320 (G4) and a second cell 324 (C9), as well as a third edge 332 that extends or connects a third gene 330 (G6) with a third cell 334 (C6). While these nodes existed before, the original data entries had indicated their gene expression level value was zero, and so no edge had connected them in first CGRG 250. With the proposed denoising process, the data has been corrected to show that for cell (C9) there is indeed gene expression of gene (G4), and similarly, for cell (C6) there is indeed gene expression of gene (G6). Thus, for purposes of this scenario, returning briefly to FIG. 2, the second zero entry 240 can be updated to the reconstructed value of 6, and the third zero entry 230 can be updated to the reconstructed value of 9. Just as importantly, the reader can further note that none of the true zeros will be changed.

As noted earlier, once data from a single-cell sequencing protocol is obtained, typically the first step in performing analyses of the data is gene filtering. As a general matter, this step is used to filter out low quality cells. A few examples of low-quality cells are doublets, cells damaged during cell isolation, or cells with too few reads to be analyzed. While there are many conventional gene filtering techniques, they rely on thresholding methods, which remove technical noise corrupted genes based on thresholds of, e.g. fold change, variance, and expression level. Unfortunately, these existing methods usually use a single fixed filtering threshold which may be over-stringent for some genes but under-stringent for others, as the amount of technical noise varies across genes. For example, using an “at least n” filter depends heavily on the choice of n. With n=10, a gene expressed in a subset of 9 cells would be filtered out, regardless of the level of expression in those cells. This may result in the failure to detect rare subpopulations that are present at frequencies below n.

Nevertheless, filtering during data analyses is essential due to the abundance of information being captured corresponding to the expression of hundreds of genes across each cell during scRNA-seq. However, only a portion of these cells will typically exhibit a reaction to the biological condition of interest, such as cell-type distinctions, differentiation-promoting factors, or responses to environmental stimuli. Due to technical noise, most of genes found in a scRNA-seq dataset will only be found at particular levels of expression. This has the effect of often making the biological signal of interest obscured by technological noise. Therefore, it is frequently desirable to select genes (filter) to exclude non-informative genes from downstream analysis. This filtering generally improves the signal-to-noise ratio in the data and, by doing so, lowers the computing complexity of down-stream data analyses.

Thus, in general, filtering can facilitate researchers in targeting their analysis to the desired “of interest” cell populations. This is meaningful because, for example, if a researcher is analyzing the behavior of different cell populations within the kidney, in many cases they do not need to keep the expression of the genes which are not expressed at all within the kidney. For example, some of these genes could be responsible for some function in the heart, brain, etc., and so are not relevant to the goal at hand.

However, when it comes to single cell sequencing that is directed to the study of abnormal tissue/samples cancer, there will not necessarily be a uniform cell population. For example, the cellular structure of a tumor is not composed of a uniform cell population, and instead has a variety of cell types. Some of these cell populations can be extremely rare yet are critical to study because the researcher may not yet know which cell population is responsible for the growth (e.g., cancer metastasis spread across all the body, with a diverse range of cell types involved, so the responsible cell population cannot be immediately recognized). In cases where the cell population that is causing metastasis is rare, or other studies involving rare cells where the outliers may be biologically relevant, the need for a careful filtering protocol becomes even more critical. Standard filtering techniques fail to offer such care, as they simply remove the genes that have low expression level values. In other words, conventional methods tend to remove low expressed genes while these genes can be very critical in discovering rare cell types in various diseases like tumors. Yet the fact that the expression level is low may indicate the data was based on a sparse and rare cell population that has a heavy impact on the target disease.

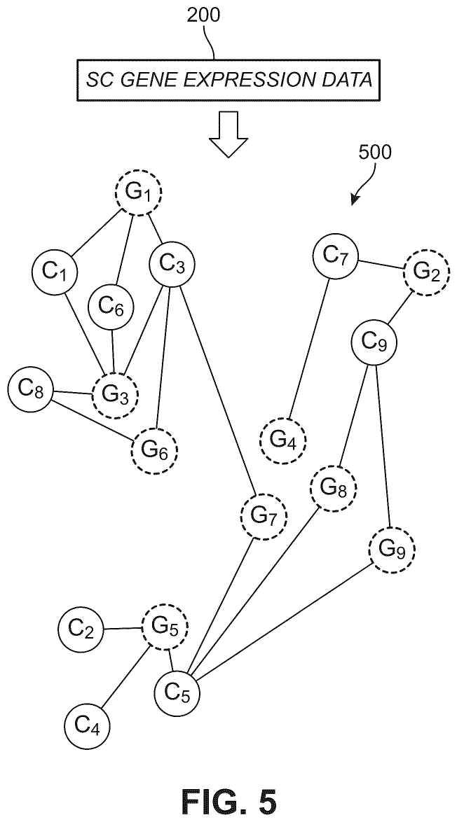

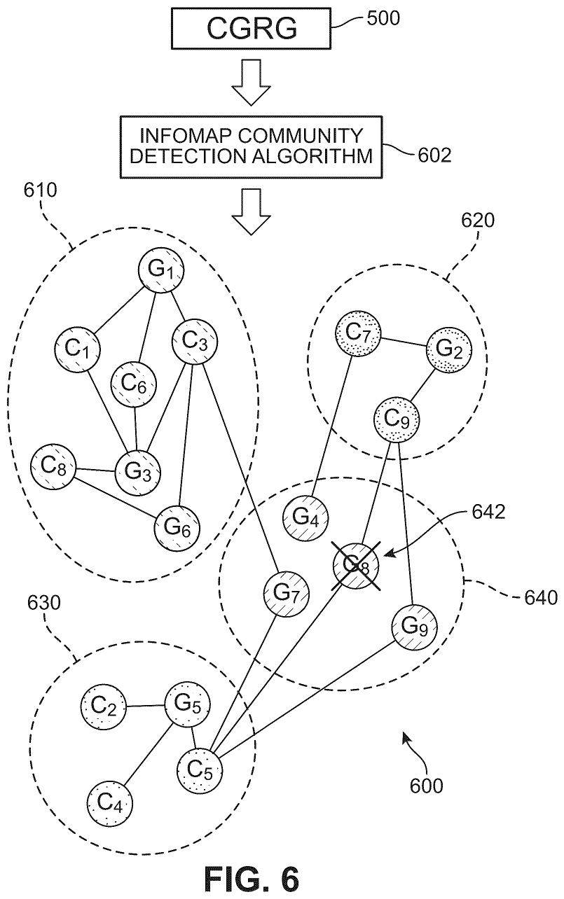

In different embodiments, the proposed filtering technique incorporates a graph mining strategy, similar to the process described above with respect to FIGS. 2-4. One example of the filtering process is now described with reference to FIGS. 5 and 6. At an initial stage, gene expression data from a single cell sequencing (e.g., data presented in single cell gene expression data matrix 200 of FIG. 2) is encoded into a single graph with nodes representing distinct entities (genes and cells) and edges denoting connections between entities. An example of this is shown in FIG. 5 by a second CGRG 500, which in this case—because it is based on the same set of data as first CGRG 250 of FIG. 2—includes the same set of nodes as first CGRG 250. In other examples with different datasets, the number of cell nodes and gene nodes can vary, as well as the edges representing the relationships between the cells and genes.

Moving now to FIG. 6, in different embodiments, in a second stage a community detection algorithm 602 (e.g., Infomap, Louvain, etc.) can be applied to the second CGRG 500. As noted earlier, the CGRG depicts objects (nodes) and connections (edges) between the objects. The connections, also called edges, can be weighted according to certain criteria such as the level of expression of the gene in a particular cell. The community detection algorithm can be used to find groups in the network with a high density of connections within and a low density of links between groups. For purposes of this disclosure, “communities” in the context of the CGRC refer to collections of cells and genes which are highly associated to each other. Such communities can presumably represent biologically significant phenotypic stability and reveal stable cellular states in the population. Hence, dividing the population into phenotypically coherent subpopulations by partitioning this graph into these communities can be of great value.

In the example of FIG. 6, a first community 610, a second community 620, a third community 630, and a fourth community 640 are depicted in an updated second CGRG 600. In different embodiments, as part of the proposed filtering process, once these communities are extracted from within the second CGRG, the weighted degree centrality for only each gene vertex (not cell vertex) in each community can be calculated. In this case, weighted degree centrality is the sum of edge weights for edges incident to the vertex.

The weighted degree centrality measurement can give greater importance to genes that are not just connected to many cells, but also have high expression levels as well. Thus, weighted degree centrality confers importance to not only the number of cells but takes into account the expression value for that specific gene as well.

However, it can be appreciated that those genes that are highly expressed in many cells across different communities might not be as informative as genes that are highly expressed in only a few communities. The proposed technique therefore offers a measure of specificity created by dividing the weighted degree centrality within a community by the weighted degree centrality across all of the communities in the graph. In different embodiments, after calculating these “specificity gene scores”, all of the genes can be ranked based on these scores. In some embodiments, the top 10% can correspond to a final selection. In FIG. 6, only one gene

(G8) has been filtered out using this process, as it has been confirmed that this gene feature is not important/significant to the expression, while the remaining gene features/nodes in the community remain.

In different embodiments, the genes can be sorted using the weighted degree centrality, where those genes in each community cluster that are below an experimentally calculated threshold can be removed. This ensures that the filtering process does not cause loss of information related to low expressed but influential genes within a rare cell population.

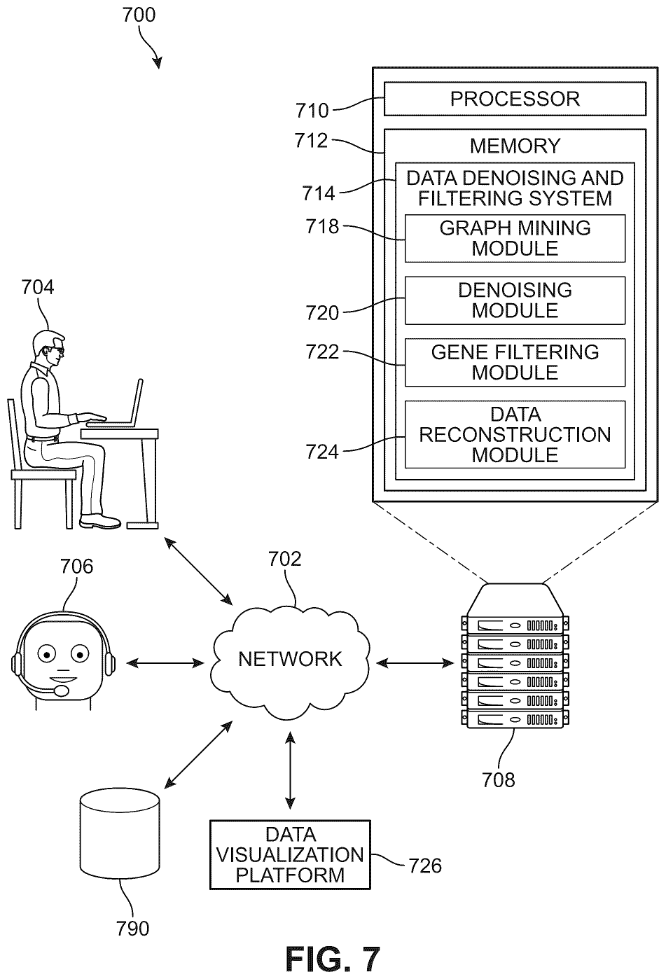

FIG. 7 is a schematic diagram of an environment 700 for an improved single cell sequencing data denoising and filtering system 714 (or system 714), according to an embodiment. The environment 700 may include a plurality of components capable of performing the disclosed methods. For example, environment 700 includes a user device 704, a computing/server system 708, a data visualization platform 726, and a database 790 which can include corrected/updated (e.g., denoised and/or filtered) data post-processing by the system 714. The components of environment 700 can communicate with each other through a network 702 and/or be linked/wired with direct connections to each other. For example, user device 704 may retrieve information from database 790 via network 702. In some embodiments, network 702 may be a wide area network (“WAN”), e.g., the Internet. In other embodiments, network 706 may be a local area network (“LAN”).

As shown in FIG. 7, components of the system 714 may be hosted in computing system 708, which may have a memory 712 and a processor 710.

Processor 710 may include a single device processor located on a single device, or it may include multiple device processors located on one or more physical devices. Memory 712 may include any type of storage, which may be physically located on one physical device, or on multiple physical devices. In some cases, computing system 708 may comprise one or more servers that are used to host the system.

While FIG. 7 shows one user device, it is understood that one or more user devices may be used. For example, in some embodiments, the system may include two or three user devices. In some embodiments, the user device may be a computing device used by a user. For example, user device 704 may include a smartphone or a tablet computer. In other examples, user device 704 may include a laptop computer, a desktop computer, and/or another type of computing device. The user devices may be used for inputting, processing, and displaying information. Referring to FIG. 7, environment 700 may further include database 790, which can include the original single cell sequencing datasets. This data may be retrieved by other components for system 714. For example, in different embodiments, system 714 may include a graph mining module 718, a denoising module 720, a gene filtering module 722, and a data reconstruction module 724. Each of these modules/components may be used to perform the operations described herein.

The method may include collecting a human tissue sample. The method may include isolating a single cell from the human tissue sample. The method may include extracting initial data from the single cell. In some embodiments, extracting initial data from a single cell includes performing single cell ribonucleic acid sequencing (scRNA-seq) on the single cell to generate first scRNA-seq data, wherein the wherein the initial data includes the first scRNA-seq data. In other words, the method may include extracting genetic material from the single cell for analysis. The data (called single cell data) produced by this process can include gene expression values of thousands of cells in the sampled tissue. In other words, the single cell data is the gene expression values representing the genetic material. In some embodiments, single cell data may be organized in a gene expression matrix with data entries representing the single cell sequencing dataset.

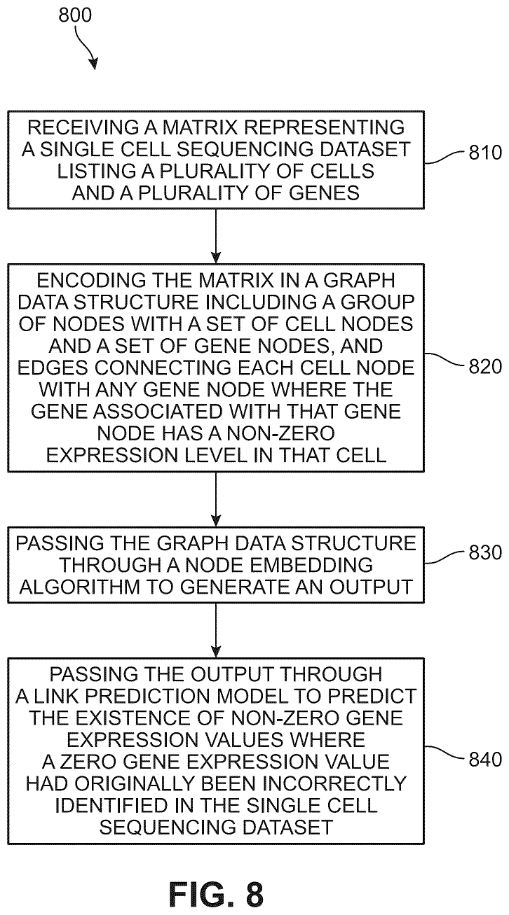

FIG. 8 is a flow chart illustrating an embodiment of a method 800 of denoising a single cell sequencing dataset. The method 800 includes a first step 810 of receiving a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell. A second step 820 includes encoding the gene expression matrix in a first graph data structure that includes a group of nodes including: (a) a set of cell nodes corresponding to the plurality of cells, (b) a set of gene nodes corresponding to the plurality of genes, and (c) edges connecting each cell node with any gene node where the gene associated with that gene node has a non-zero expression level in the cell associated with that cell node. In addition, a third step 830 includes passing the first graph data structure through a node embedding algorithm to compute an output including a vector representation of each node and each edge, and a fourth step 840 includes passing the output through a link prediction model to predict the existence of non-zero gene expression values where a zero gene expression value had originally been incorrectly identified in the single cell sequencing dataset.

In other embodiments, the method may include additional steps or aspects. In some embodiments, a first data entry for a first gene had a zero gene expression value for a first cell in the gene expression matrix, and the method further includes determining, via the link prediction model, that the first gene has a non-zero expression level within the first cell. In some embodiments, the method also includes reconstructing the single cell sequencing dataset to correct the data entries with zero gene expression values that the link prediction model determined were actually non-zero, including the first data entry. In another embodiment, the first graph data structure includes a first cell node corresponding to the first cell and a first gene node corresponding to the first gene and the method further includes adding an additional edge to the first graph data structure that connects the first cell node with the first gene node to produce a second graph data structure. In different embodiments, a majority of the data entries in the gene expression matrix includes a zero gene expression value that is either a biological zero that is a true absence of a gene's expression in a cell or a non-biological zero artificially introduced during the generation of the single cell sequencing dataset, and the reconstruction retains the zero gene expression values data entries that reflect biological zeros. In some embodiments, the method further includes passing the first graph data structure through a community detection algorithm to identify a group of communities of more densely interconnected nodes. In some embodiments, the method also includes calculating a specificity gene score for each gene node to generate a set of specificity gene scores; and excluding those gene nodes with a specificity gene score less than at least 75% of the set of specificity gene scores.

Other methods may be contemplated within the scope of the present disclosure. For example, in some embodiments, a method of improving accuracy of gene filtering and reducing technical noise in single cell sequencing datasets is disclosed. The method includes a first step of receiving a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell. A second step includes encoding the gene expression matrix in a first graph data structure that includes a group of nodes including: (a) a set of cell nodes corresponding to the plurality of cells, (b) a set of gene nodes corresponding to the plurality of genes, and (c) edges connecting each cell node with any gene node where the gene associated with that gene node has a non-zero expression level in the cell associated with that cell node. A third step includes passing the first graph data structure through a community detection algorithm to identify a group of communities of more densely interconnected nodes. In addition, a fourth step includes calculating a specificity gene score for each gene node to generate a set of specificity gene scores, and a fifth step includes excluding those gene nodes with a specificity gene score less than at least 75% of the set of specificity gene scores.

In other embodiments, the method may include additional steps or aspects. In some embodiments, the method includes computing, for each vertex of a gene node in each community of the group of communities, a weighted degree centrality measurement; and computing, for all communities, a total weighted degree centrality value, where calculating the specificity gene score for each gene node includes dividing the weighted degree centrality measurement for the vertex of that gene node by the total weighted degree centrality value. In another embodiment, the plurality of cells was taken from a metastatic tumor. In some embodiments, the method also includes passing the first graph data structure through a node embedding algorithm to compute an output including a vector representation of each node and each edge. In different embodiments, the method further includes passing the output through a link prediction model to predict the existence of non-zero gene expression values where a zero gene expression value had originally been incorrectly identified in the single cell sequencing dataset. In one embodiment, a first data entry for a first gene had a zero gene expression value for a first cell in the gene expression matrix, and the method further includes determining, via the link prediction model, that the first gene has a non-zero expression level within the first cell.

While the disclosed embodiments are discussed with the application of analyzing single cells, including RNA of cells, it is understood the disclosed embodiments can also be used with other applications. For example, the disclosed systems and methods can be used in other types of analysis involving complex networks.

Embodiments may include a non-transitory computer-readable medium (CRM) storing software comprising instructions executable by one or more computers which, upon such execution, cause the one or more computers to perform the disclosed methods. Non-transitory CRM may refer to a CRM that stores data for short periods or in the presence of power such as a memory device or Random Access Memory (RAM). For example, a non-transitory computer-readable medium may include storage components, such as, a hard disk (e.g., a magnetic disk, an optical disk, a magneto-optic disk, and/or a solid-state disk), a compact disc (CD), a digital versatile disc (DVD), a floppy disk, a cartridge, and/or a magnetic tape.

Embodiments may also include one or more computers and one or more storage devices storing instructions that are operable, when executed by the one or more computers, to cause the one or more computers to perform the disclosed methods.

Certain embodiments may use cloud computing environments. Cloud computing environments can include, for example, an environment that hosts the services for impact analysis and detection described herein. The cloud computing environment may provide computation, software, data access, storage, etc. services that do not require end-user knowledge of a physical location and configuration of system(s) and/or device(s) that hosts the impact analysis and detection services. For example, a cloud computing environment may include a group of computing resources (referred to collectively as “computing resources” and individually as “computing resource”).

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some examples be excised from the combination, and the claimed combination may be directed to a sub-combination or variation of a sub-combination.

We claim:

1. A method of denoising a single cell sequencing dataset, the method comprising:
receiving a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell;
encoding the gene expression matrix in a first graph data structure including:
a group of nodes including:
a set of cell nodes corresponding to the plurality of cells, and
a set of gene nodes corresponding to the plurality of genes, and
edges connecting each cell node with any gene node where the gene associated with that gene node has a non-zero expression level in the cell associated with that cell node;
passing the first graph data structure through a node embedding algorithm to compute an output including a vector representation of each node and each edge; and
passing the output through a link prediction model to predict the existence of non-zero gene expression values where a zero gene expression value had originally been incorrectly identified in the single cell sequencing dataset.

2. The method of claim 1, wherein a first data entry for a first gene had a zero gene expression value for a first cell in the gene expression matrix, and the method further comprises determining, via the link prediction model, that the first gene has a non-zero expression level within the first cell.

3. The method of claim 2, further comprising reconstructing the single cell sequencing dataset to correct the data entries with zero gene expression values that the link prediction model determined were actually non-zero, including the first data entry.

4. The method of claim 2, wherein the first graph data structure includes a first cell node corresponding to the first cell and a first gene node corresponding to the first gene and the method further comprises adding an additional edge to the first graph data structure that connects the first cell node with the first gene node to produce a second graph data structure.

5. The method of claim 3, wherein a majority of the data entries in the gene expression matrix include a zero gene expression value that is either a biological zero that is a true absence of a gene's expression in a cell or a non-biological zero artificially introduced during generation of the single cell sequencing dataset, and the reconstruction retains the zero gene expression values data entries that reflect biological zeros.

6. The method of claim 1, further comprising passing the first graph data structure through a community detection algorithm to identify a group of communities of more densely interconnected nodes.

7. The method of claim 6, further comprising:
calculating a specificity gene score for each gene node to generate a set of specificity gene scores; and
excluding those gene nodes with a specificity gene score less than at least 75% of the set of specificity gene scores.

8. A method for improving accuracy of gene filtering and reducing technical noise in single cell sequencing datasets, the method comprising:
receiving a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell;
encoding the gene expression matrix in a first graph data structure including:
a group of nodes including:
a set of cell nodes corresponding to the plurality of cells, and
a set of gene nodes corresponding to the plurality of genes, and
edges connecting each cell node with any gene node where the gene associated with that gene node has a non-zero expression level in the cell associated with that cell node;
passing the first graph data structure through a community detection algorithm to identify a group of communities of more densely interconnected nodes;
calculating a specificity gene score for each gene node to generate a set of specificity gene scores; and
excluding those gene nodes with a specificity gene score less than at least 75% of the set of specificity gene scores.

9. The method of claim 8, further comprising:
computing, for each vertex of a gene node in each community of the group of communities, a weighted degree centrality measurement; and
computing, for all communities, a total weighted degree centrality value,
wherein calculating the specificity gene score for each gene node includes dividing the weighted degree centrality measurement for the vertex of that gene node by the total weighted degree centrality value.

10. The method of claim 8, wherein the plurality of cells was taken from a metastatic tumor.

11. The method of claim 8, further comprising passing the first graph data structure through a node embedding algorithm to compute an output including a vector representation of each node and each edge.

12. The method of claim 11, further comprising passing the output through a link prediction model to predict the existence of non-zero gene expression values where a zero gene expression value had originally been incorrectly identified in the single cell sequencing dataset.

13. The method of claim 12, wherein a first data entry for a first gene had a zero gene expression value for a first cell in the gene expression matrix, and the method further comprises determining, via the link prediction model, that the first gene has a non-zero expression level within the first cell.

14. A system for denoising a single cell sequencing dataset comprising one or more computers and one or more storage devices storing instructions that are operable, when executed by the one or more computers, to cause the one or more computers to:
receive a gene expression matrix with data entries representing the single cell sequencing dataset, the gene expression matrix listing a plurality of cells and a plurality of genes, where for each gene in the plurality of genes, the matrix lists a gene expression value that indicates that gene's measured expression level within each cell;
encode the gene expression matrix in a first graph data structure including:
a group of nodes including:
a set of cell nodes corresponding to the plurality of cells, and
a set of gene nodes corresponding to the plurality of genes, and
edges connecting each cell node with any gene node where the gene associated with that gene node has a non-zero expression level in the cell associated with that cell node;
pass the first graph data structure through a node embedding algorithm to compute an output including a vector representation of each node and each edge; and
pass the output through a link prediction model to predict the existence of non-zero gene expression values where a zero gene expression value had originally been incorrectly identified in the single cell sequencing dataset.

15. The system of claim 14, wherein a first data entry for a first gene had a zero gene expression value for a first cell in the gene expression matrix, and the instructions further cause the one or more computers to determine, via the link prediction model, that the first gene has a non-zero expression level within the first cell.

16. The system of claim 15, wherein the instructions further cause the one or more computers to reconstruct the single cell sequencing dataset to correct the data entries with zero gene expression values that the link prediction model determined were actually non-zero, including the first data entry.

17. The system of claim 15, wherein the first graph data structure includes a first cell node corresponding to the first cell and a first gene node corresponding to the first gene, and the instructions further cause the one or more computers to add an additional edge to the first graph data structure that connects the first cell node with the first gene node to produce a second graph data structure.

18. The system of claim 16, wherein a majority of the data entries in the gene expression matrix include a zero gene expression value that is either a biological zero that is a true absence of a gene's expression in a cell or a non-biological zero artificially introduced during generation of the single cell sequencing dataset, and the reconstruction retains the zero gene expression values data entries that reflect biological zeros.

19. The system of claim 14, wherein the instructions further cause the one or more computers to pass the first graph data structure through a community detection algorithm to identify a group of communities of more densely interconnected nodes.

20. The system of claim 14, wherein the instructions further cause the one or more computers to:
calculate a specificity gene score for each gene node to generate a set of specificity gene scores; and
exclude those gene nodes with a specificity gene score less than at least 75% of the set of specificity gene scores.
